(19) United States
(12) Patent Application Publication
(10) Pub. No.: US 2021/0338454 A1
(43) Pub. Date: Nov. 4, 2021

(54) 3D PRINTED OSTEOGENESIS SCAFFOLD

(71) Applicant: MCA Group, LLC, La Jolla, CA (US)

(72) Inventor: Thomas Afzal, La Jolla, CA (US)

(21) Appl. No.: 17/323,764

(22) Filed: May 18, 2021

Related U.S. Application Data
(63) Continuation-in-part of application No. 17/224,870, filed on Apr. 7, 2021.
Application No. 17/224,870 is a continuation of application No. 16/711,091, filed on Dec. 11, 2019, now abandoned.
Application No. 16/711,091 is a continuation of application No. 15/947,620, filed on Apr. 6, 2018, now Pat. No. 10,507,118.
Application No. 15/947,620 is a continuation of application No. 15/299,347, filed on Oct. 20, 2016, now abandoned.
(60) Provisional application No. 62/244,374, filed on Oct. 21, 2015.
(60) Provisional application No. 63/027,181, filed on May 19, 2020.

(51) Int. Cl. A61F 2/44; A61B 17/72

(52) U.S. Cl. CPC A61F 2/4465; A61F 2002/30062; A61F 2/4455; A61B 17/72

(57) ABSTRACT

Osteogenesis scaffold such as for spinal fusion or an intermedullary nail includes a number of arcuate struts. The scaffold may have a functional modulus of elasticity that is a result of the modulus of the material of the struts together with the architecture of the struts, and may be within the range of 5 GPa and 75 GPa. An anisotropy of a physical property such as stiffness, compressive strength or elastic modulus corresponds to the same physical property of native bone in the vicinity of the intended implantation site.

### CROSS-REFERENCE TO RELATED APPLICATIONS

This application is a continuation-in-part of Ser. No. 17/224,870, filed Apr. 7, 2021, which is a continuation application of U.S. application Ser. No. 16/711,091, filed Dec. 11, 2019, which is a continuation application of U.S. application Ser. No. 15/947,620, filed Apr. 6, 2018, now U.S. Pat. No. 10,507,118, which is a continuation application of U.S. application Ser. No. 15/299,347, filed Oct. 20, 2016, which claims the priority benefit under 35 U.S.C. § 119(e) of U.S. Provisional Application No. 62/244,374, filed Oct. 21, 2015, the entireties of which are hereby incorporated by reference herein. This application also claims the priority benefit under 35 U.S.C. § 119(e) of U.S. Provisional Application No. 63/027,181, filed May 19, 2020.

### BACKGROUND OF THE INVENTION

Spondylosyndesis, or spinal fusion, is a surgical technique used to combine two or more vertebrae into a single, rigid working unit. This is typically achieved by introducing a supplementary bone tissue, such as an autograft or allograft, into the intervertebral space between two target vertebrae, at the location that is typically occupied by an intervertebral disc. The supplementary bone tissue is then used in conjunction with the patient's natural osteoblastic processes in order to grow bone or osseous tissue between the two or more target vertebrae, which acts to fuse them together into the desired rigid unit. This procedure is used primarily to eliminate pain that is caused by abnormal motion of one or both of the target vertebrae; pain relief occurs by immobilizing the vertebrae themselves and preventing the abnormal motion. Alternatively, surgically implantable synthetic intervertebral fusion cages or devices may be used to perform spinal fusion procedures.

Surgically implantable intervertebral fusion cages are well known in the art and have been actively used to perform spinal fusion procedures for many years. Their use became popularized during the mid 1990's with the introduction of the BAK Device from the Zimmer Inc. The BAK system is a fenestrated, threaded, cylindrical, titanium alloy device that is capable of being implanted into a patient as described above through an anterior or posterior approach, and is indicated for cervical and lumbar spinal surgery. Most common spinal fusion systems today are made from metals, such as titanium or cobalt chrome alloys, or from a polymer such as polyetheretherketone (PEEK) which is commonly used in biomedical implants. Unfortunately, these implant materials have a modulus which is much higher than that of bone and there is clinical evidence of implant subsidence and movement which is believed to be attributable to mechanical incompatibility between natural bone and the implant material. Also bone pressure necrosis does occur as a result of the presence of these metal implants.

Implants based on bone material from a donor (allograft) or from the patient itself (autograft) do have an inconsistent mechanical strength and show subsidence over time. The inconsistent properties of these implants make them generally unpredictable, challenging to reliably machine and especially prone to migration and explusion due to the difficulty of consistently machining teeth into the upper and lower implant contact surfaces.

Although titanium alloy cages give good fusion rates, their modulus is significantly dissimilar to human bone. The stress transfer between an implant device and a bone is not homogeneous when Young's moduli of the implant device and the bone are different. This results in stress shielding. In such conditions, bone atrophy occurs and leads to the loosening of at the implant bone interface and eventually lead to failure. Therefore, the stiffness (Young's modulus) of the implant is preferably not too high compared to that of bone. Implant devices made from metallic biomaterials such as stainless steels, Co—Cr alloys, and titanium (Ti) and its alloys have a Young's modus generally much greater than that of the bone. Young's moduli of the most widely used stainless steel for implant devices, SUS316L stainless steel and Co—Cr alloys, are around 180 GPa and 210 GPa, respectively. Young's moduli of Ti (pure titanium) and its alloys are generally smaller than those of stainless steels and Co—Cr alloys. For example, Ti and its alloy, Ti-6Al-4V ELI, which are widely used for constructing implant devices, have a Young's modulus of around 110 GPa. However, this value is still higher than that of the bone, which is on the order of 10-30 GPa.

The foregoing shortcomings in the spinal fusion cage arts apply to other orthopedic implants as well, such as intermedullary nails for long bones such as the femur.

Therefore, there remains a need for a biostable implant such as for use as an orthopedic implant or plate which has a tensile modulus comparable to that of bone, which does not subside and provides good stability.

### SUMMARY OF THE INVENTION

There is provided in accordance with one aspect of the invention, an osteogenesis scaffold configured for sacroiliac fusion. The osteogenesis scaffold comprises an elongate body having a proximal end, a distal end and a triangular transverse cross section; a matrix of arcuate struts on the body providing the body with at least one functional modulus; wherein the struts are configured to give the body a first modulus at a midpoint between the proximal end and the distal end, that approximates the modulus of cortical bone, and a second, lower modulus at the proximal end and distal end, that approximates the modulus of cancellous bone.

A transverse cross sectional area through the body may decrease as the cross section approaches at least one of the proximal and distal ends. The first modulus may be about 14 to about 16 GPa. The second modulus may be about 2 to about 3 GPa.

There is also provided an osteogenesis scaffold configured for spinal fusion. The osteogenesis scaffold comprises a superior support surface; an inferior support surface; and a plurality of arcuate struts separating the superior and inferior support surfaces, the struts comprising a material having a strut modulus; the scaffold having a functional modulus which is different than the strut modulus and is the result of the strut modulus and the architecture of the implant.

The functional modulus may be within the range of from about 5 GPa to about 75 GPa. The functional modulus may be within the range of from about 10 GPa to about 30 GPa. The functional modulus in a first portion of the implant may be different than the functional modulus in a second portion of the implant.

The osteogenesis scaffold may comprise a lumbar cage, having a peripheral modulus within the range of from about 7 GPa to about 16 GPa. The lumbar cage may be configured for use with normal bone, having a peripheral modulus within the range of from about 14 GPa to about 16 GPa. The functional modulus in a periphery of the superior support surface may be greater than the functional modulus in the center of the superior support surface. The functional modulus in a periphery of the superior support surface may be at least about 300% greater than the functional modulus in the center of the superior support surface. The functional modulus in a periphery of the superior support surface may be at least about 500% greater than the functional modulus in the center of the superior support surface.

There is also provided a proximal femoral osteogenesis scaffold, comprising a head post for receiving a femoral head; a shoulder; and a femoral stem connected to the shoulder; wherein an exterior surface of the shoulder comprises a smooth surface and the surface of the femoral stem comprises a matrix of struts. The femoral stem has a proximal end in the direction of the shoulder and a distal end, and an increased flexibility in the distal direction.

### BRIEF DESCRIPTION OF THE DRAWINGS

FIG. 1 is a side elevational perspective view of an implant in accordance with the present invention, in the form of a spinal fusion cage.

FIG. 2 is a top plan view of the spinal fusion cage shown in FIG. 1.

FIG. 3 is a perspective elevational cross section through the spinal cage of FIG. 2.

FIG. 4 is an elevational cross section through the cage of FIG. 2, taken along an axis perpendicular to the cross-section of FIG. 3.

FIG. 5 is a side elevational view of the cage of FIG. 1.

FIG. 6A is a perspective view of another embodiment of a spinal fusion cage.

FIG. 6B is a front view of the spinal fusion cage of FIG. 6A.

FIG. 6C is a top view of the spinal fusion cage of FIG. 6A.

FIG. 6D is a cross-sectional view of the spinal fusion cage of FIG. 6C through line A-A.

FIG. 6E is a perspective view of a posterior fragment as seen in FIG. 6D.

FIG. 6F is a perspective view of an anterior fragment as seen in FIG. 6D.

FIG. 7 is a perspective view of another embodiment of a spinal fusion cage.

FIG. 8A is a perspective view of another embodiment of a spinal fusion cage.

FIG. 8B is a front view of the spinal fusion cage of FIG. 8A.

FIG. 8C is a top view of the spinal fusion cage of FIG. 8A.

FIG. 8D is a cross-sectional view of the spinal fusion cage of FIG. 8C.

FIG. 9A is a perspective view of another embodiment of a spinal fusion cage.

FIG. 9B is a front view of the spinal fusion cage of FIG. 9A.

FIG. 9C is a top view of the spinal fusion cage of FIG. 9A.

FIG. 9D is a cross-sectional view of the spinal fusion cage of FIG. 9C through line A-A.

FIG. 10A is a perspective view of another embodiment of a spinal fusion cage.

FIG. 10B is a front view of the spinal fusion cage of FIG. 10A.

FIG. 10C is a top view of the spinal fusion cage of FIG. 10A.

FIG. 10D is the cross-sectional view of the spinal fusion cage of FIG. 10C through line D-D.

FIG. 10E is an enlarged fragmentary view of a portion of FIG. 10D

FIG. 10F is a perspective view of a fragment as seen in FIG. 10E.

FIG. 11 is a perspective view of another embodiment of a spinal fusion cage.

FIG. 12 is a perspective view of another embodiment of a spinal fusion cage.

FIG. 13A is a perspective view of an osteogenesis scaffold.

FIG. 13B is the top view of the osteogenesis scaffold of FIG. 13A.

FIG. 13C is an end view of the osteogenesis scaffold of FIG. 13A.

FIG. 13D is the cross-sectional view of the osteogenesis scaffold of FIG. 13B through line X-X.

FIG. 13E is a detailed view of the structure of the osteogenesis scaffold of FIG. 13D.

FIG. 14A is a perspective view of another osteogenesis scaffold.

FIG. 14B is a side elevational view of the osteogenesis scaffold of FIG. 14A.

FIG. 14C is a top plan view of the osteogenesis scaffold of FIG. 14A.

FIG. 14D is a cross-sectional view of the osteogenesis scaffold of FIG. 14C through line E-E.

FIG. 15A is a perspective view of another embodiment of an osteogenesis scaffold.

FIG. 15B is a top plan view of the osteogenesis scaffold of FIG. 15A.

FIG. 15C is a side view of the osteogenesis scaffold of FIG. 15A.

FIG. 15D is a cross-sectional view of the osteogenesis scaffold of FIG. 15C through line X-X.

FIG. 15E is a bottom view of the osteogenesis scaffold of FIG. 15A.

FIG. 15F is an end view of the osteogenesis scaffold of FIG. 15A.

### DETAILED DESCRIPTION OF THE PREFERRED EMBODIMENT

The present invention addresses the problem by providing implants such as intermedullary nails, fusion plates or spinal fusion cages that have a functional modulus of elasticity that is substantially the same as the modulus of elasticity of the native bone at the implant site. For example, implants can be provided having a functional modulus of elasticity of between about 5 GPa and about 75 GPa, typically between about 10 GPa and about 50 GPa and in some implementations between about 10 and about 30 GPa. More specifically, implants can be provided having a functional modulus of elasticity in a first portion of the implant of between about 20 GPa and about 40 GPa, typically between about 25 GPa and about 35 GPa and in some implementations between about 28 and about 32 GPa. The same implant can be provided having a functional modulus of elasticity of in a second, different portion of the implant between about 5 GPa and about 30 GPa, typically between about 10 GPa and about 20 GPa and in some implementations between about 13 and about 17 GPa.

Certain specific implementations are described below. A lumbar cage, intended for normal bone, may have a peripheral modulus of about 14-16 or about 15 GPa and a center modulus of about 2-3 or about 2.5 GPa. A lumbar cage, intended for osteopenic bone may have a peripheral modulus of about 10-12 or 11 GPa and a center of about 0.5-2 or about 1 GPa. A lumbar cage, intended for osteoporotic bone may have a peripheral modulus of about 7-9 or about 8 GPa and a center modulus of about 0.3-1.5 or about 0.7 GPa. In a cervical fusion cage, each of the above values may be increased by about 10% or 15%. Thus the peripheral modulus may be at least about 6 GPa or 8 GPa or 10 GPa or more, greater than the center modulus in the same implant, depending upon the desired performance.

Functional modulus means the effective modulus of the final implant, which will be the result of both the modulus of the material of the implant as well as the result of the arcuate strut architecture of the implant, as will be described below.

In addition, human or animal tissue is generally not structurally isotropic. For example, cancellous bone includes trabeculae, also referred to as spicules, defining a plurality of open spaces. The trabeculae and open spaces are generally oriented in a direction of principle stress (e.g., axially along a long bone such as a femur). The trabeculae form a porous or spongy-type tissue that is generally stiffer in a particular direction. For example, cancellous bone in the femur is generally stiffer axially than radially to accommodate an axial direction of the primary stress on the bone.

A prosthetic orthopedic implant such as a fusion cage should therefore be designed to avoid producing load concentrations which can lead to stress shielding of nearby bone. Bone can remodel to adapt to the load applied to it. If a particular location within a bone experiences increased load, the body will increase bone growth at that location. The reverse is also true. In response to a reduced load at a particular location, the body will tend to resorb bone from that location. Therefore, concentrating stresses within a prosthetic implant providing structural support can lead to weakening and resorption of the surrounding bone.

An isotropic implant together with the anisotropy of nearby bone, can lead to stress shielding, such as if the isotropic implant is stiffer in one direction (e.g., in a radial direction, for a long bone such as a femur) than the nearby native bone. An anisotropic porous scaffold support structure can help reduce or avoid such stress shielding, such as by providing anisotropy in a similar direction to the anisotropy of the nearby native bone.

For example, the osteogenesis scaffold can be configured so that an anisotropy of a physical property, such as stiffness, compressive strength, elastic modulus, and the like, is the same or substantially the same as an anisotropy of the same physical property in the native bone in the vicinity of the intended implantation. In an example, the porous scaffold can be configured to be stiffer in a first direction (e.g., axially) compared to a second direction (e.g., laterally), such as to mimic anisotropic stiffness of nearby native bone in the first direction and second direction. The porous scaffold can be configured so that the physical property, such as stiffness, is the same or substantially the same as the same physical property in the nearby native bone in both the first direction and the second direction. In an example, “substantially the same,” when referring to the matching of a physical property between the porous scaffold and the nearby native bone can refer to the value of the physical property of the porous scaffold in the first direction being within about 10% or preferably within about 5% of the value of the physical property of the nearby native bone in the first direction, such as within 3%, 1%, or less. Similarly, the physical property in the second direction can be considered to be substantially the same if the value of the physical property of the porous scaffold in the second direction is within about 10% or preferably within about 5%, or within 3%, 1%, or less of the value of the physical property of the nearby native bone in the second direction.

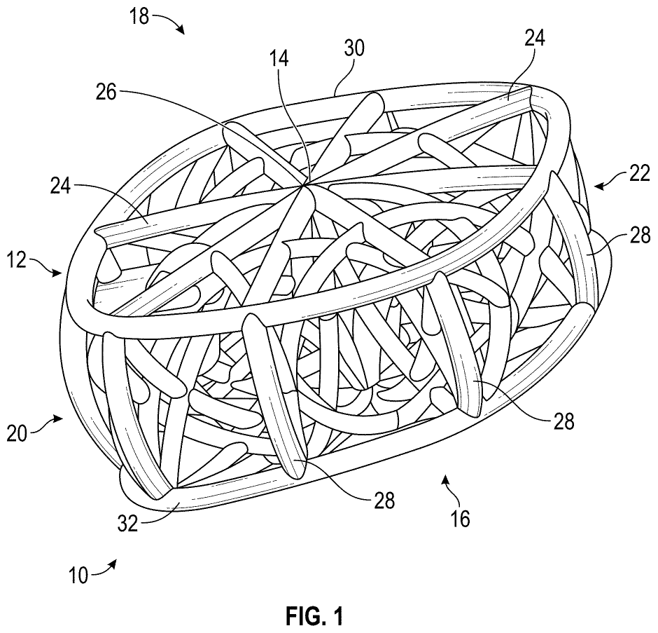

Referring to FIG. 1, there is illustrated a perspective view of an osteogenesis scaffold 10. The scaffold 10 can take any of a variety of configurations depending upon the intended anatomical environment, and is illustrated in FIG. 1 in the form of a spinal fusion cage 12.

Fusion cage 12 comprises a superior support surface 14, and an inferior support surface 16 spaced apart by a body portion 18. Measured in an axial direction, the anterior side 20 typically has a greater height then a posterior side 22.

The osteogenesis cage 12 comprises a plurality of arcuate struts, configured to produce an implant having a functional modulus which is a composite of the modulus of the material of construction, taken together with the physical properties attributable to the architecture of the implant. In the illustrated embodiment, the cage 12 comprises a plurality of arcuate struts configure to permit a degree of compression and expansion in the axial (superior inferior) direction, in response to cyclic physiologic load. Each arcuate strut is configured to function as a leaf spring, within the constraints imposed by the material and geometry of the struts.

In the illustrated embodiment, the superior support surface 14 comprises a plurality of struts as will be discussed. Alternatively, the superior support surface 14 and or inferior support service 16 may comprise a unitary apertured or porous plate or other construct for engaging the adjacent bony end plate.

In the illustrated embodiment, the superior support surface 14 comprises a plurality of interior surface struts 24 extending radially outwardly from a centerpoint 26 along the superior support surface 14. At least about two or four or six or eight or more interior surface struts 24 may be provided. In the illustrated embodiment, four long struts intersect at the centerpoint 26 to provide eight interior surface struts 24.

The surface struts 24 described above may be reproduced on the inferior support surface 16 in a symmetrical arrangement. The surface struts 24 may reside in a plane. Preferably, however, the surface struts 24 define an arcuate surface which is slightly convex in a direction away from the body 18, to complement the surface of the bony end plate of the adjacent vertebral body.

The superior support service 14 is spaced apart from the inferior support surface 16 by, among other things, a plurality of peripheral axial struts 28. In the illustrated embodiment, each of the radially outwardly facing ends of the surface struts 24 is connected to an axial strut 28. Thus, in the illustrated embodiment, eight axial struts 28 are positioned about the periphery of the body 18. However, any of a variety of numbers such as at least about four, six, eight, 10, 12 or more axial struts 28 maybe provided depending upon the overall desired scaffold design. Axial struts 28 maybe linear, or, preferably, each axial strut 28 may define an arc. In the illustrated embodiment, each of the axial struts 28 is concave in the direction of the central axis of the body 18. This may allow a slight axial compression of the body 18 under anatomical loads.

The intersections of the axial struts 28 and surface struts 24 are connected by a superior peripheral frame 30. In the illustrated embodiment, the peripheral frame 30 comprises a continuous annular strut, defining the outer periphery of the superior support surface 14. The inferior support surface 16 is provided with a symmetrical peripheral frame 32, defining the outer periphery of the inferior support surface 16.

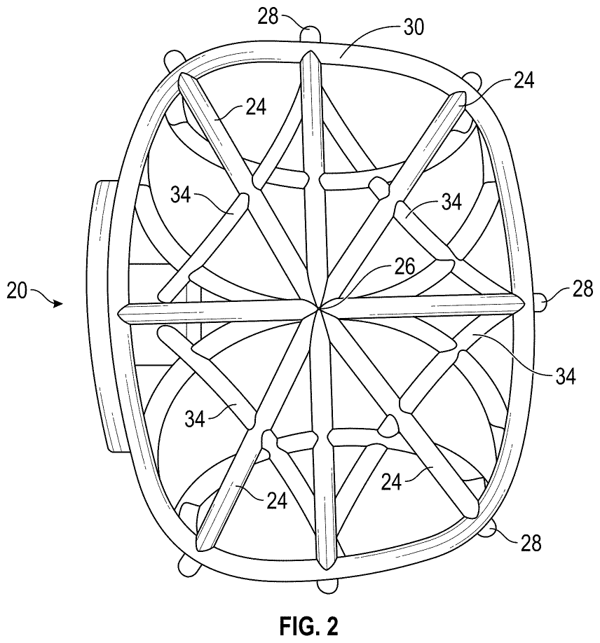

Referring to FIG. 2, there is illustrated a top plan view of the superior support surface 14. A plurality of diagonal surface struts 34 join radial ends of alternating radial surface struts 24. Diagonal surface struts 34 may be nonlinear, and, in the illustrated embodiment, are arcuate with a concavity pointing in the direction of the periphery of the body 18. At least two or four or more diagonal surface struts 34 may be provided in each plane such as the superior support surface 14. In the illustrated embodiment, four diagonal service struts 34 are provided, intersecting the peripheral frame 30 at approximately 90° spacing.

The strut geometry residing in the plane of the superior support surface 14 may be symmetrically reproduced for the inferior support surface 16. That strut geometry may be further reproduced within one or two or more intermediate planes, residing in between the superior support surface 14 and inferior support surface 16.

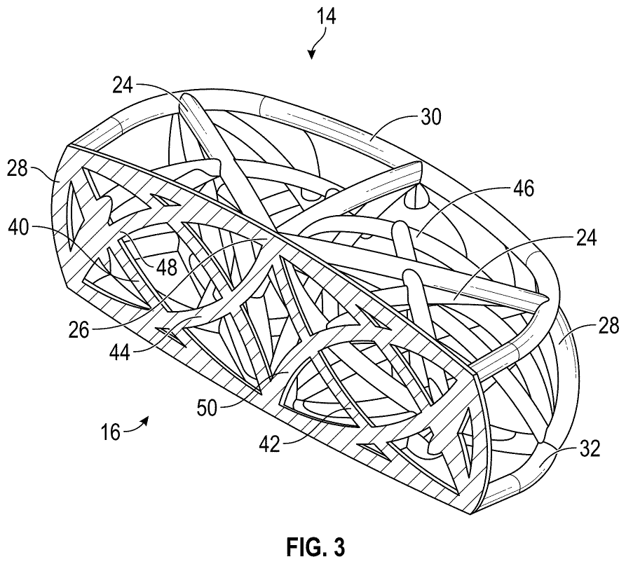

Referring to FIG. 3, there is illustrated a vertical cross-section through a central surface strut 24. Within the body 18, a plurality of struts are provided. At least about 50%, preferably least about 80%, and typically at least about 90 or 95% of the struts are curved.

The first concave upward strut 40 extends from the peripheral frame 30 on the superior support surface 14, to the inferior support surface 16, and back to a second end of the peripheral frame 30 on superior support surface 14. A second concave upward strut 42 extends from the inferior frame 32 to the centerpoint 26 on the superior support surface 14. Each of the first second and third concave upward struts have an arcuate configuration with an upward facing concavity.

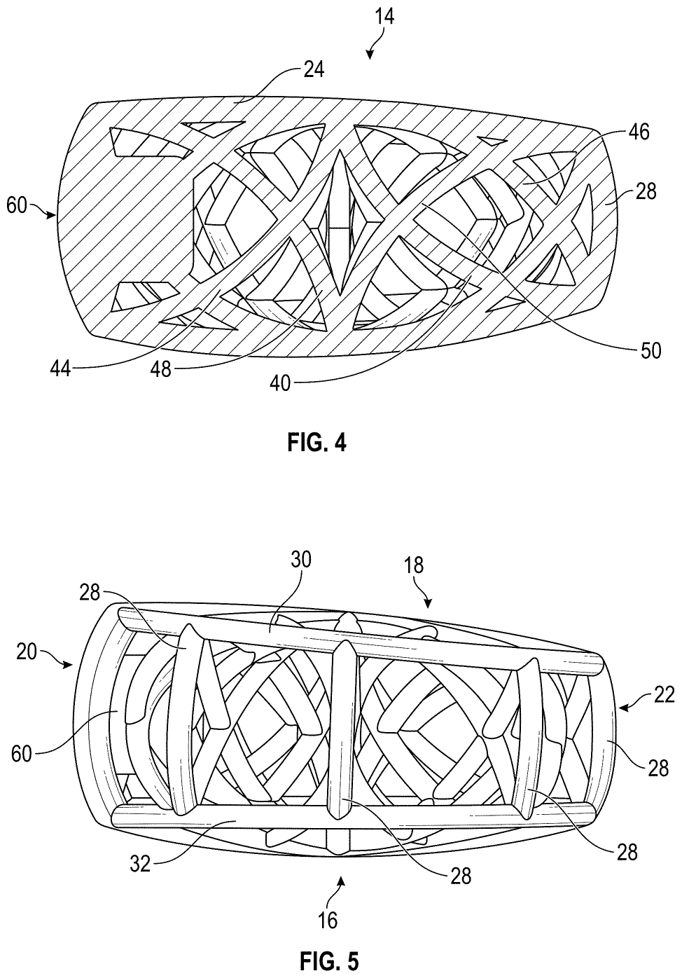

Referring to FIGS. 4 and 5, the peripheral surface of the body 18 is provided with a dock 60, for releasable engagement with an insertion tool. The dock 60 may be provided with an aperture, projection, or other surface structure (not illustrated) which is complementary to a distal portion of an insertion tool. For example, the dock 60 may be provided with a threaded aperture for threadable engagement with a threaded distal end of an insertion tool. The dock 60 is preferably provided on a peripheral surface of the implant, and maybe on the posterior, anterior, lateral or posterior lateral sides, depending upon the desired route of implantation.

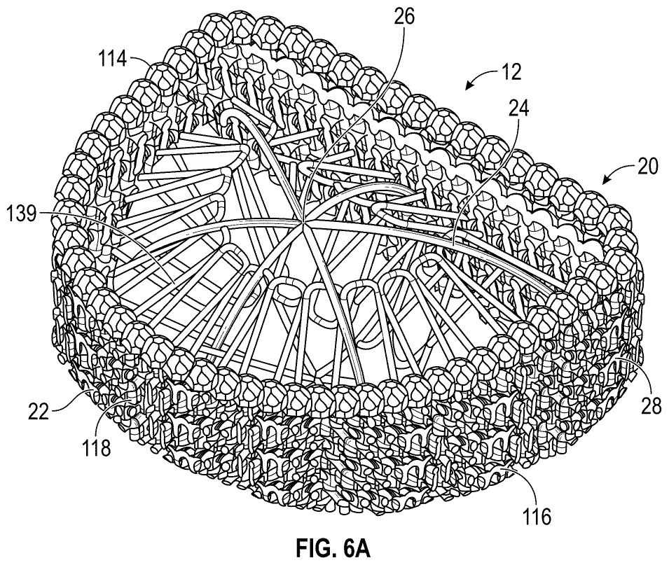

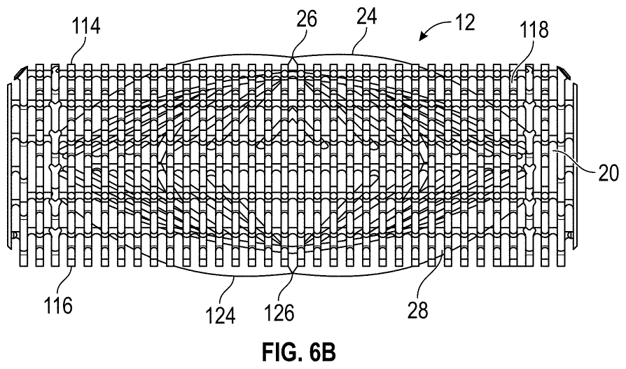

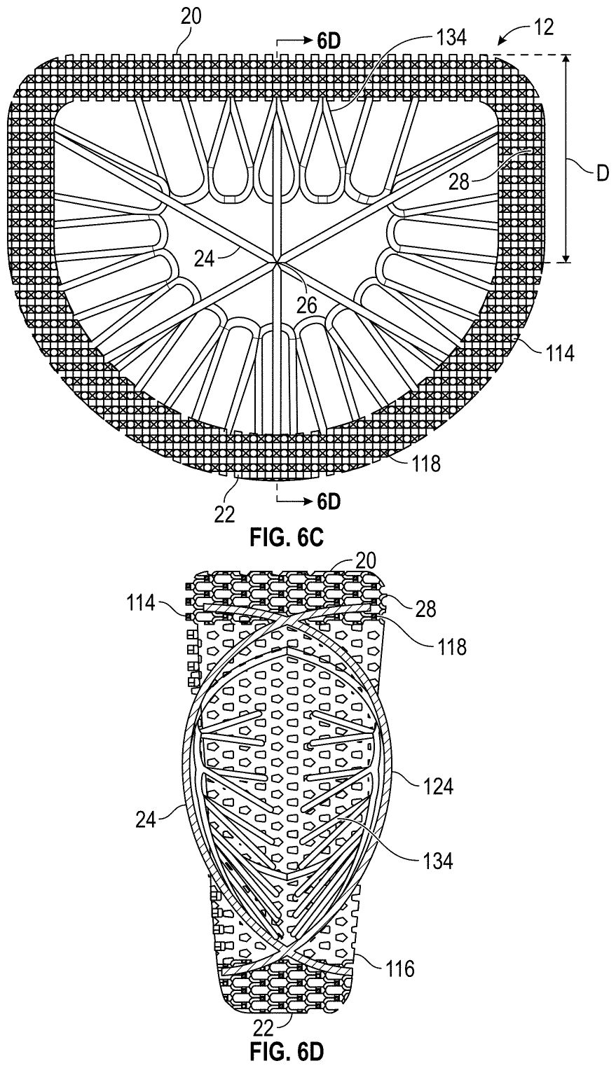

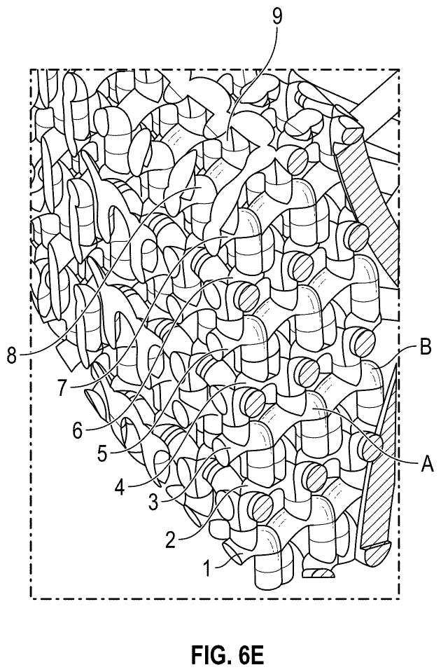

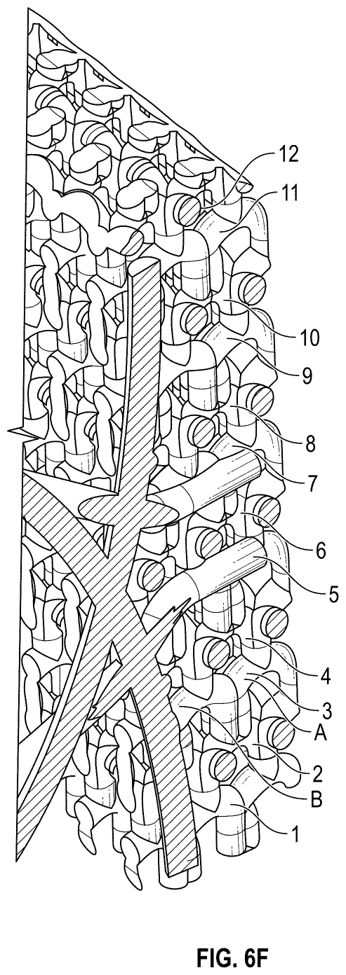

FIGS. 6A-6D show an embodiment of the spinal fusion cage 12 with a superior perimeter support surface 114, inferior perimeter support surface 116, and an anterior side 20 that typically has a greater height than a posterior side 22. The posterior side 22 is generally curved to match the posterior curve of the patient's vertebral body. The anterior side 20 can have a relatively flat face to act as a support surface for an insertion instrument that is used to insert the spinal fusion cage 12 as is described above.

The superior perimeter support surface 114 and inferior perimeter support surface 116 are connected by a matrix of arcuate struts 118 that are stacked in multiple vertical rows. In this embodiment, 3 layers of 6 vertical rows (anterior side and 4 vertical rows posterior side) are stacked around the entire perimeter between the superior perimeter support surface 114, inferior perimeter support surface 116. This matrix or plurality of arcuate struts is configured to permit a degree of compression and expansion in the axial (superior-inferior) direction is response to physiologic load. Each arcuate strut is configured to function as a leaf spring and the composite matrix of arcuate struts 118 is configured to have an elastic modulus that matches the modulus of the cortical bone around the perimeter of the vertebral body.

Within the matrix of arcuate struts 118 are arcuate surface struts 24 that extend from a center point 26 to the inside of the matrix of arcuate struts 118. These surface struts 24 form a slightly convex superior surface in the direction away from composite matrix of arcuate struts 118. There is a comparable set of surface struts 124 form a slightly convex inferior surface in the direction away from composite matrix of arcuate struts 118. In addition to the arcuate surface struts 24 and 124 that extend from center points 26 and 126 respectively, there is a plurality of diagonal arcuate struts 134 that extend from the inside of the matrix of arcuate struts 118 to a superior and inferior surface.

Where the matrix of arcuate struts 118 are configured with an elastic modulus that matches the modulus of the cortical bone around the perimeter of the vertebral body, the arcuate surface struts 24 and 124 and plurality of diagonal arcuate struts 134 are also configured to function as a leaf springs and to make contact with and have a modulus of elasticity that matches the modulus of the cancellous bone that in in the center of the vertebral body. This variable modulus of elasticity from the stiffer matrix of arcuate struts 118 to the more flexible arcuate surface struts 24 and 124 and plurality of diagonal arcuate struts 134 that is configured to match the cortical and cancellous vertebral bone modulus respectively provides optimal bone remodeling to and within the spinal fusion cage 12.

For example, measured in the vertical, transverse to the vertebral body endplate, the modulus measured around the periphery of the implant is no more than about 18 GPa and in some implementations no more than about 8 GPa depending upon the condition of the native bone. The modulus measured approximately at the center of the end plate is generally at least about 6 GPa and in some embodiments at least about 0.7 GPa. A normal endplate may be within the range of from about 6 to 2.5 GPa, an osteopenic endplate may be within the range of from about 2.5 to 1 GPa and an osteoporotic endplate may be within the range of from about 0.1 to 0.5 GPa. The modulus may transition gradually between the center of the end plate and the peripheral wall. In general, the peripheral modulus will be at least about 300%, 500%, or 1000% of the central modulus.

FIGS. 6E and 6F are detailed figures to show the layers of arches. The posterior (FIG. 6E) has 9 layers of arches high (1-9) that are 2+ layers thick (A-B and partial layers) and the anterior (FIG. 6F) has 12 layers of arches high (1-12) that are 2+ layers thick (A-B and partial layers).

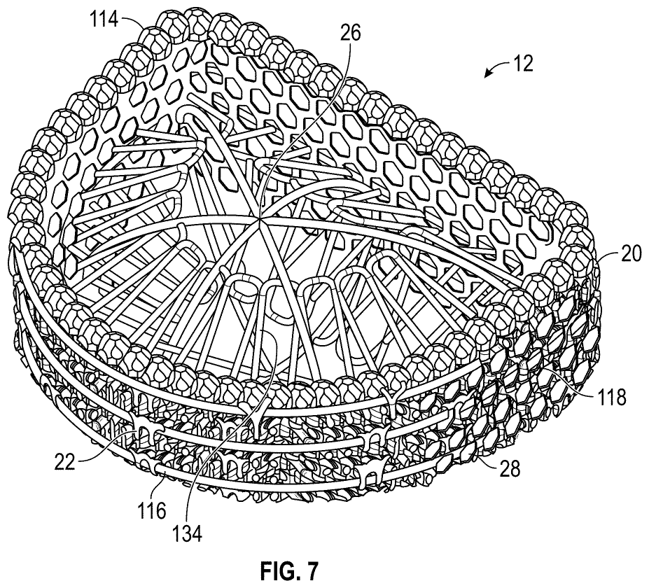

Referring now to FIG. 7 an embodiment of the spinal fusion cage 12 with a matrix of arcuate struts 118 is shown, wherein the matrix of arcuate struts 118 is configured with just 2 layers of 5 vertical rows (anterior side and 3 vertical rows posterior side) that are stacked around the entire perimeter between the superior perimeter support surface 114, inferior perimeter support surface 116. This configuration of the matrix of arcuate struts 118 is comprised of arcuate struts that are longer and have a greater radius of arc compared to the matrix of arcuate struts 118 shown in FIG. 6A. This less dense matrix of arcuate struts 118 provides a more elastic modulus for the perimeter of the spinal fusion cage 12 that would match the modulus of cortical bone in a patient with less dense vertebral bodies.

By varying arc length and arc diameter and there for the resulting modulus of the matrix of arcuate struts 118 the spinal fusion cage 12 can be configured to have an elasticity that matches either a patient with “normal” bone or one with osteopenic bone or one with osteoporotic bone. In a similar manner the arcuate surface struts 24 and 124 and plurality of diagonal arcuate struts 134 can be vary in arc length, arc diameter, and number of arcs so that the spinal fusion cage 12 can be configured with an interior modulus that matches the cancellous bone of a patient with “normal” bone or one with osteopenic bone or one with osteoporotic bone. In addition, the vertical inclination of the arcs can be changed to change the elasticity of the spinal fusion cage 12. In FIGS. 6A-6D and FIG. 7, the diagonal arcuate struts 134 in the central portion of the are angled relative to the vertical direction. This angle can be varied to change the elastic modulus of that portion of the spinal fusion cage 12, where in the greater the angle from vertical the lower the resulting modulus created by the diagonal arcuate struts 134.

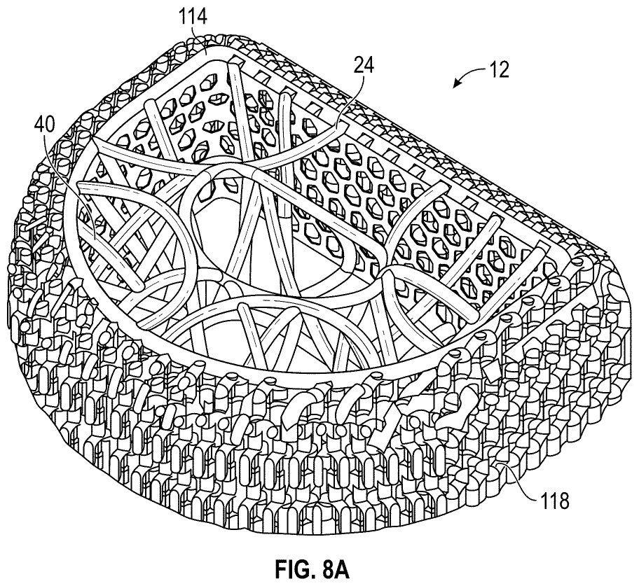

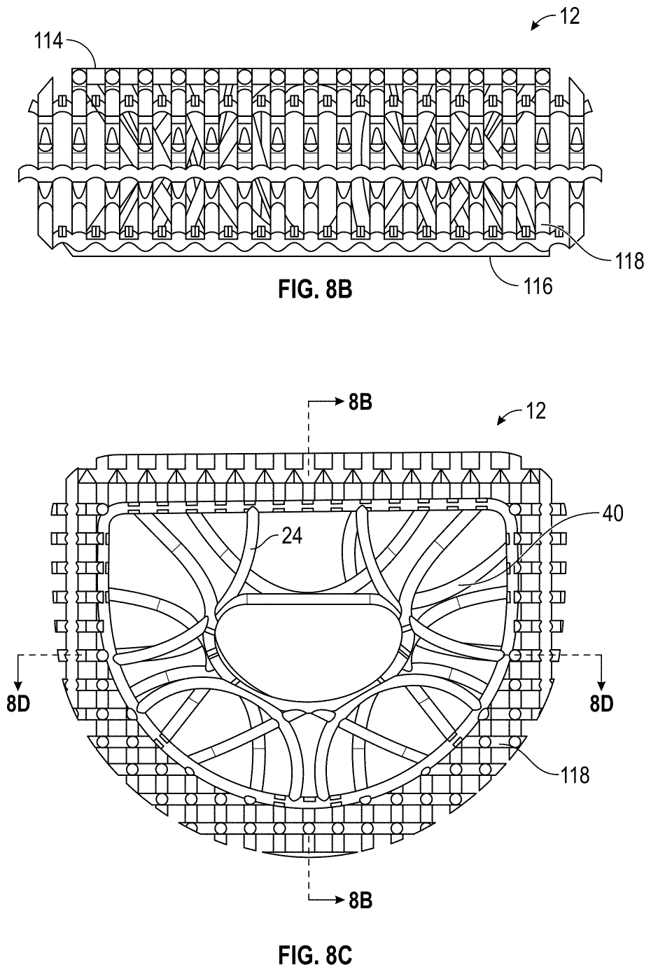

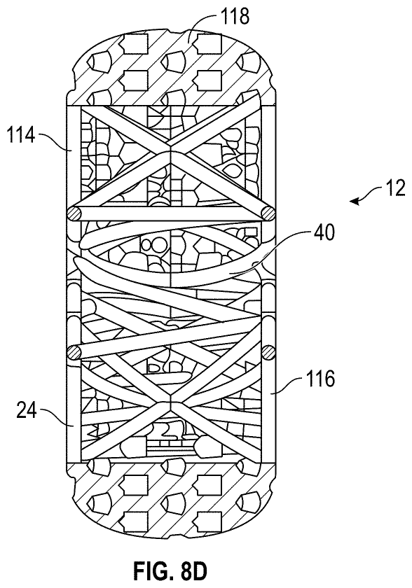

Referring to FIGS. 8A-8D an embodiment of the spinal fusion cage 12 with a matrix of arcuate struts 118 is shown, wherein the matrix of arcuate struts 118 is configured with just 3 layers of 5 vertical rows that are stacked around the entire perimeter between the superior perimeter support surface 114, inferior perimeter support surface 116. The thickness of the arcs in the matrix of arcuate struts 118 is greater than the thickness of the arcs shown in FIGS. 6A-6D and FIG. 7. Varying the thickness of the arcs will vary the modulus of the matrix of arcuate struts 118 just as varying the arc length and arc diameter varies the modulus. An increased thickness will increase the modulus and a decreased thickness will decrease the modulus. The arcuate surface struts 24 on the superior surface in this embodiment of the spinal fusion cage 12 are in the same plane as the superior perimeter surface 114.

This embodiment is configured to engage with vertebral bodies that are relatively flat without a concave portion. As well the superior perimeter surface 114 and the inferior perimeter surface 116 are relatively parallel. This embodiment of the spinal fusion cage 12 is configured to engage vertebral bodies that have little or no lordosis between them such as the more superior lumbar levels. This embodiment is also comprised of several concave upward arcuate struts 40 as were described earlier herein. The number and configuration of concave upward struts 40 and the arcuate surface struts 24 are such that the center of the spinal fusion cage 12 has more open area for bone graft and bone ingrowth than some of the previously described embodiments.

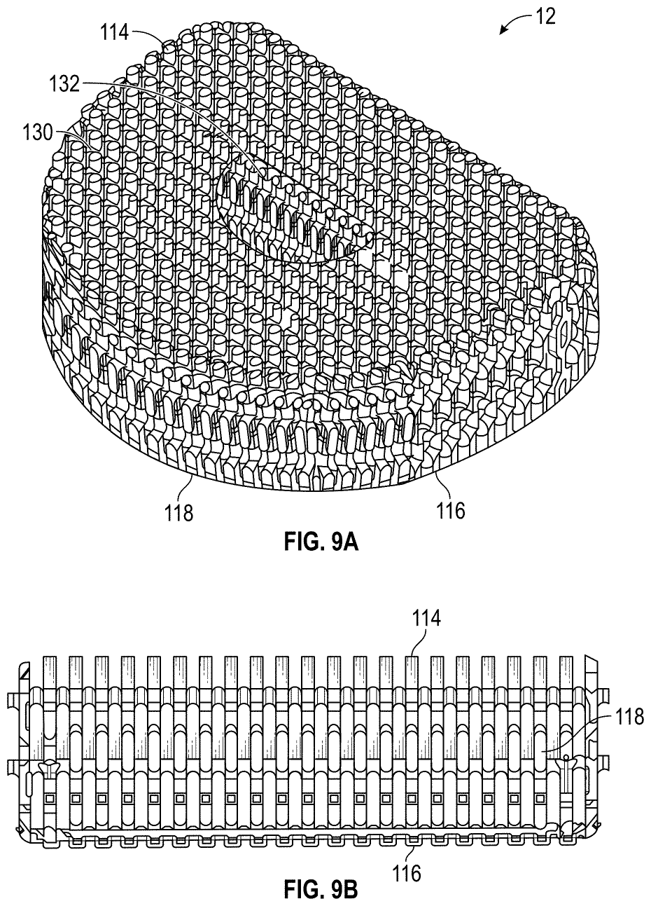

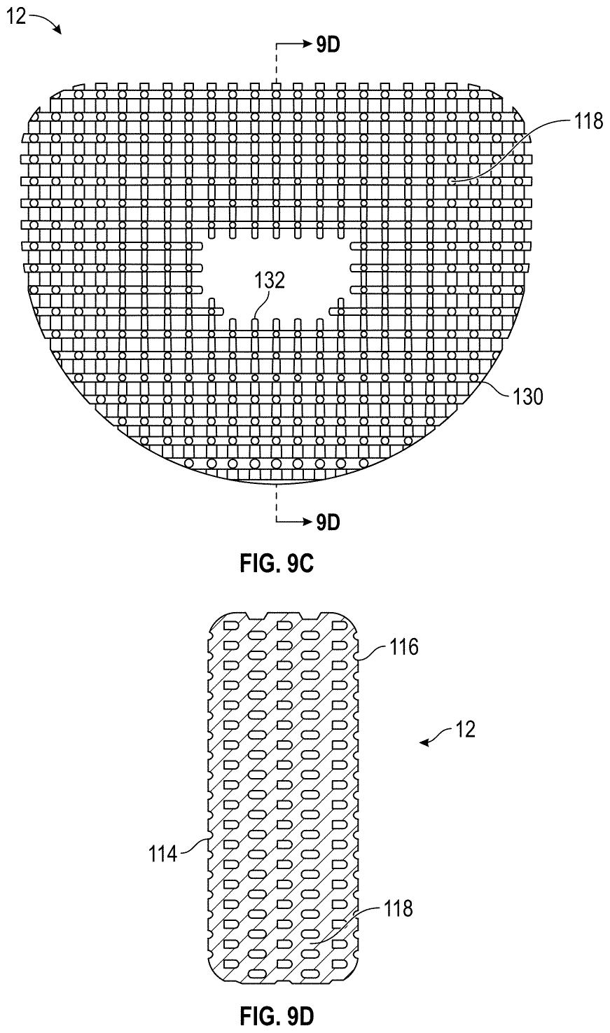

Referring to FIGS. 9A-9D an embodiment of the spinal fusion cage 12 with a matrix of arcuate struts 118 is shown, wherein the matrix of arcuate struts 118 is configured to form the entirety of the spinal fusion cage 12. The thickness of the arcs in the matrix of arcuate struts 118 vary from thick on the perimeter 130 to thin in the center 132 of the spinal fusion cage 12. With this configuration the modulus of the spinal fusion cage 12 gradually varies from stiffer on the perimeter 130 to more flexible at the center 132 as the thickness of the arcs gradually decrease. This arc thickness variation can be configured such that the modulus of the perimeter 130 and the modulus of the center 132 matches the modulus of the cortical bone on the perimeter of the vertebral body and the modulus of the cancellous bone in the center of the vertebral body. As well the length and diameter of the arcs can be varied as was previously described herein along with the arc thickness to create the modulus variation that matches the bone modulus.

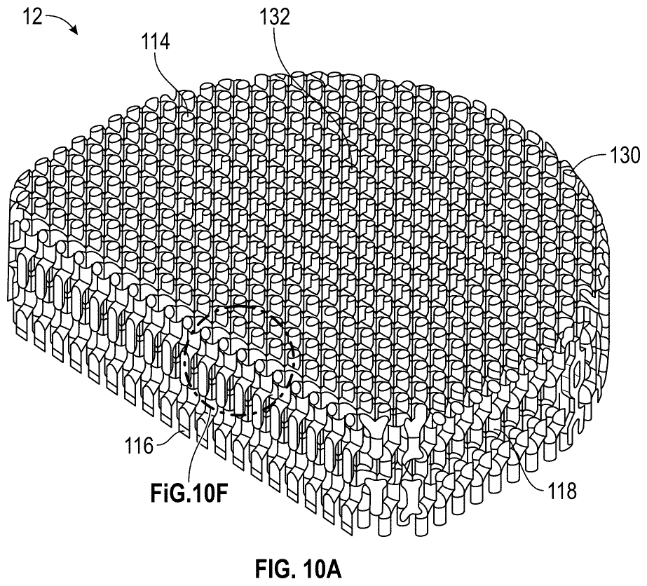

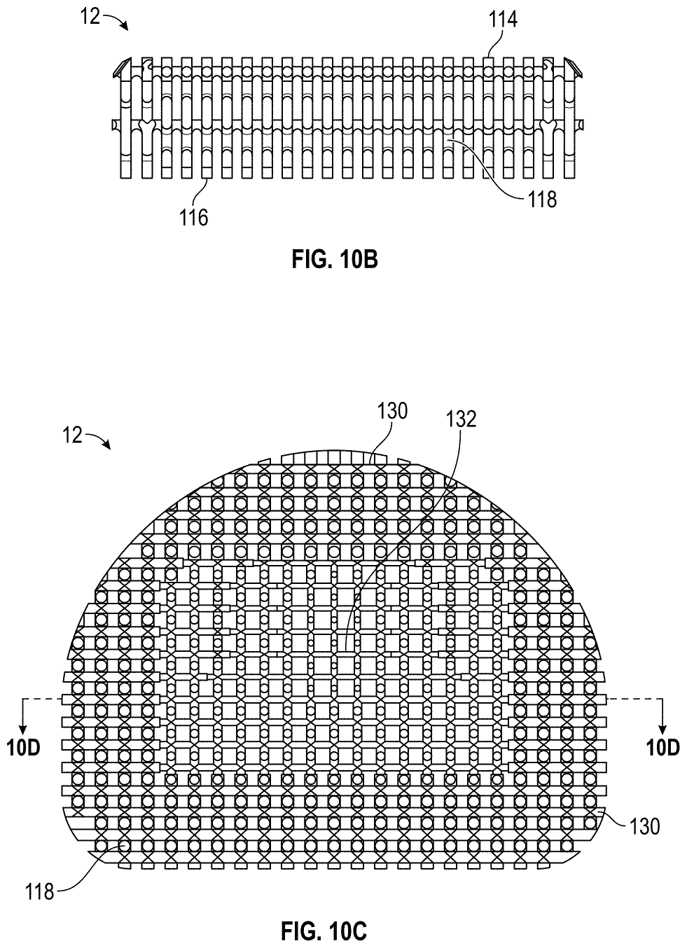

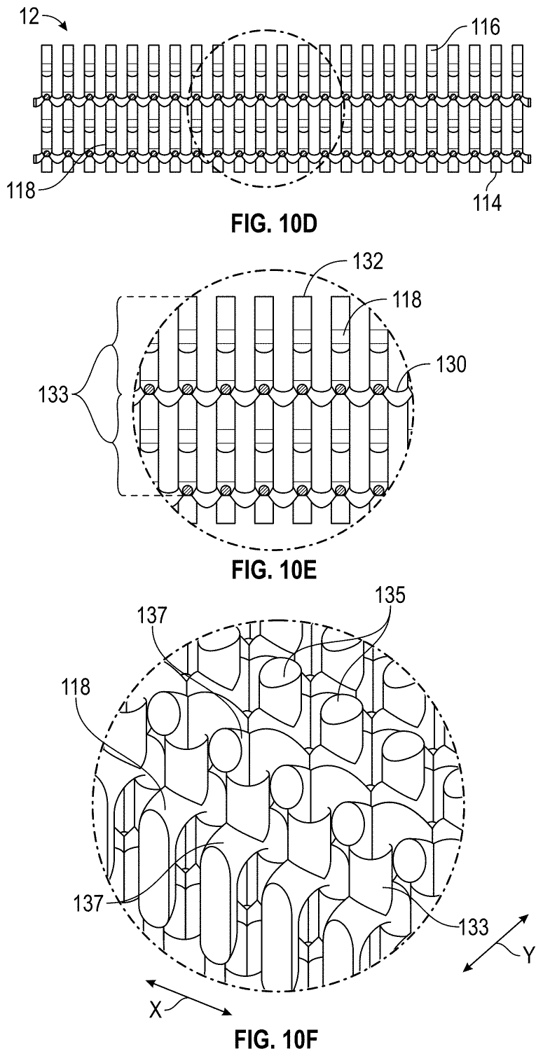

Referring to FIGS. 10A-10H an embodiment of the spinal fusion cage 12 with a matrix of arcuate struts 118 is shown, wherein the matrix of arcuate struts 118 is configured to form the entirety of the spinal fusion cage 12. In this embodiment the matrix of arcuate struts 118 creates a spinal fusion cage 12 without an axial opening in the center. This configuration provides for contact of the entire superior surface 14 and inferior surface 16 with the vertebral body surface. Maximizing contact of the surfaces of the spinal fusion cage 12 with the vertebral bodies is useful to provide the cyclic loading between the spinal fusion cage 12 and the vertebral bodies to optimize bone growth and healing. The thickness of the arcs in the matrix of arcuate struts 118 vary from thick on the perimeter 130 to thin in the center 132 of the spinal fusion cage 12 to vary the spinal fusion cage 12 modulus as was previously described. The density of the matrix of arcuate struts 118 can be configured to provide both maximum contact with the vertebral body and enough porosity for bone ingrowth and healing without the need for a central axial opening.

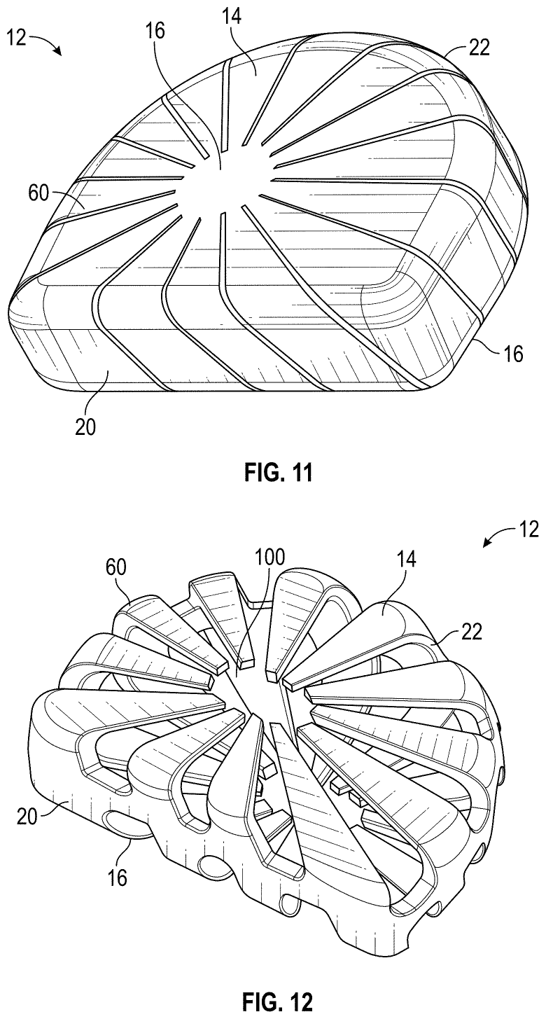

Referring to FIG. 11 an embodiment of the spinal fusion cage 12 is shown with a superior support surface 14, an inferior support surface 16, an anterior side, and a posterior side 22. The spinal fusion cage 12 is comprised of arcuate struts 60 that extend from a center point 26 across the superior support surface 14 to the perimeter then curve down to the inferior support surface 16 and then curve again back to another center point on the inferior support surface 16. To provide a spinal fusion cage 12 that matches the modulus of the bone, the thickness of these arcuate struts 60 can be varied to be thinner near the center point 26 and thicker where they curve down from the superior support surface 14 to the inferior support surface 16.

Referring to FIG. 12 an embodiment of the spinal fusion cage 12 is shown with a superior support surface 14, an inferior support surface 16, an anterior side, and a posterior side 22. The spinal fusion cage 12 is comprised of arcuate struts 60 that extend from a center opening 100 across the superior support surface 14 to the perimeter then curve down to the inferior support surface 16 and then curve again back to another center point on the inferior support surface 16. To provide a spinal fusion cage 12 that matches the modulus of the bone, the thickness of these arcuate struts 60 can be varied to be thinner near the center point 26 and thicker where they curve down from the superior support surface 14 to the inferior support surface 16. The width of the arcuate struts 60 can also be varied to provide a variation in modulus. The width of the arcuate struts 60 can be thinner towards the center opening 100 and thicker towards the perimeter. Varying the width of the arcuate struts 60 also provides for the maximum contact of the superior support surface 14 and the inferior support surface 16 of the spinal fusion cage 12 with the vertebral body while also providing enough opening for bone ingrowth and healing.

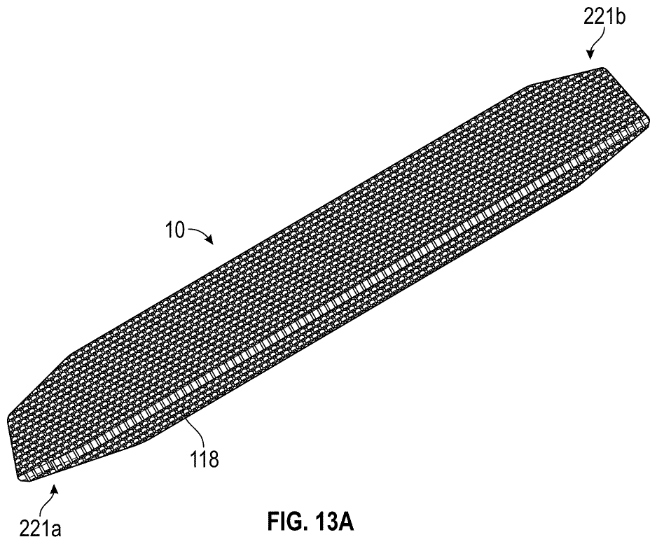

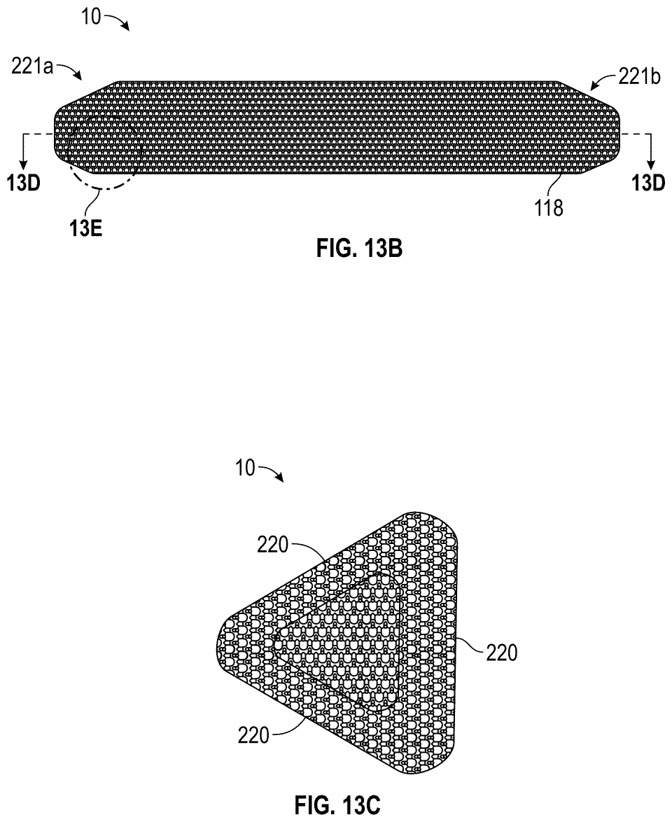

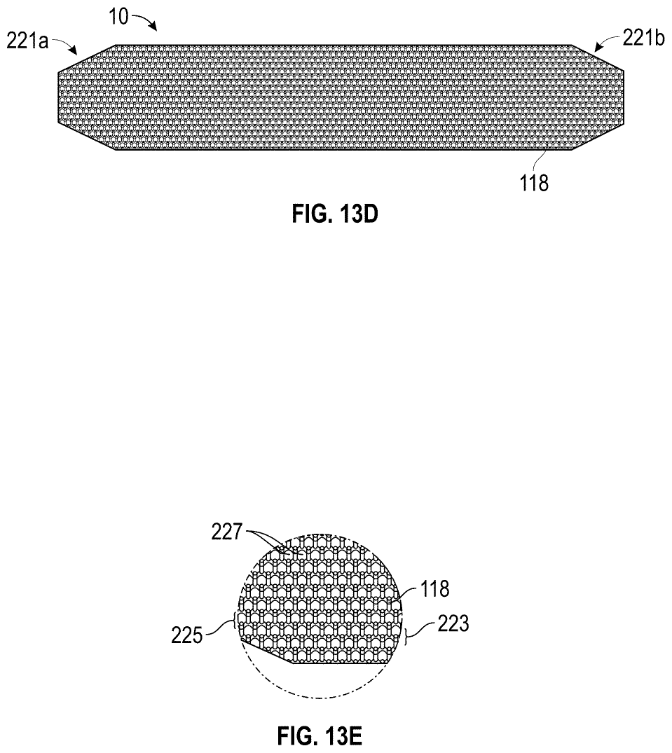

FIGS. 13A-13E show an embodiment of an osteogenesis scaffold 10 comprising matrix of arcuate struts 118. The matrix of arcuate struts 118 forms a three-sided prism 220 with tapered ends 221a and 221b, The prism shape and tapered ends 221a and 221b provide an osteogenesis scaffold 10 that is shaped for insertion in a surgically formed bore traversing from a patient's sacrum across the sacroiliac joint and into their ilium. This osteogenesis scaffold 10 is useful for patients with degenerated sacroiliac joints where fusing the sacrum to the ilium prevents relative movement between these two structures and thereby reduces the patient's pain.

In FIG. 13E a detail of the matrix of arcuate struts 118 is shown where a one row of struts 223 is layered above another row of struts 225 with each row offset from the previous row. This structure provides a very strong support as well as cavities 227 to facilitate bone ingrowth and fusion.

Although the osteogenesis scaffold 10 shown in FIGS. 13A-13E are shown with a uniform matrix of arcuate struts 118, it is possible to vary the matrix by making the strut cross-section thinner from the midpoint towards the tapered ends 221a and 221b. It is also possible to vary the matrix by making the density of struts vary from high density at the midpoint to lower density at the tapered ends 221a and 221b. It is also possible to vary the matrix by making the height and arch length of the struts vary from shorter height and smaller arch length at the midpoint to taller height and larger arc length at the tapered ends 221a and 221b.

As was described earlier, the angle of inclination of the matrix relative to the vertical direction can also be varied to modify the modulus of the matrix. Either one or any combination of these four variations provides an osteogenesis scaffold 10 that is stiffer at the midpoint and more flexible towards the tapered ends 221a and 221b. Varying the osteogenesis scaffold 10 stiffness is important as the osteogenesis scaffold 10 needs to provide a solid fusion of the sacrum to the ilium at the midpoint which is the junction of two cortical bones, but needs to be more flexible at the tapered ends 221a and 221b that are imbedded in the sacrum or the ilium in the cancellous bone such that the stiffness of the tapered ends matches the stiffness of the more flexible cancellous bone. This variable stiffness will help prevent unwanted fractures of the more flexible cancellous bone that can occur when a fusion implant with constant stiffness is used.

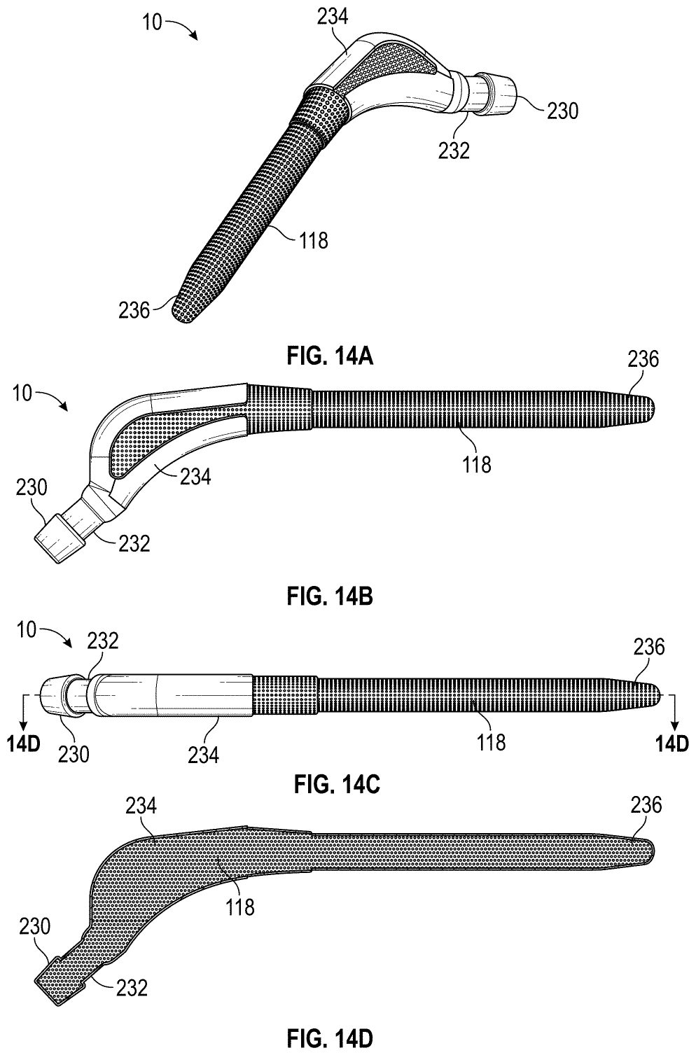

Referring now to FIGS. 14A-14D an embodiment of the osteogenesis scaffold 10 with a matrix of arcuate struts 118 is shown. The matrix of arcuate struts 118 is configured in the shape of a hip replacement femoral stem for placement in a patient's femur. It is comprised of a head post 230, a neck 232, a shoulder 234 and a long-tapered stem 236. The head post 230 is configured to receive a spherical or other shaped femoral head that provides the articulating joint surface with the opposing acetabular cup. The neck 232 is recessed to ensure full articulation of the acetabular cup relative to the femoral stem. The shoulder 234 transfers loads from the head post 230 to the long-tapered stem 236 and into the patient's femur.

The head post 230, a neck 232, and shoulder 234 are comprised of a generally smooth solid exterior and either a solid interior or an interior matrix of arcuate struts 118. The long-tapered stem 236 is comprised of a matrix of arcuate struts 118. A common problem for existing hip replacement implants is secondary bone fractures of the femur due to the stem of the implant in the femur being too stiff relative to the femur bone. As it is comprised of a matrix of arcuate struts 118, the osteogenesis scaffold 10 can have a stiffness modulus that is stiffer in the head post 230, a neck 232, and shoulder 234 and increasing flexibility in the distal direction along the long-tapered stem 236. In this manner, the osteogenesis scaffold 10 can be more rigid at the interface with the femoral head and more flexible in the distal intramedullary interface with the patient's femur.

As was previously described herein, the variation in stiffness can be achieved using variations in arcuate strut thickness, height, arc length and/or density. As well the matrix of arcuate struts 118 in the long-tapered end 236 provides for bone ingrowth as was previously described herein. For example, the modulus of the head post 230 can be no more than about 10 GPa or can be as low as 7 GPa, and the modulus of the neck 232 can be no more than about 5 Gpa or can be as low as 3 GPa. Likewise the modulus of the shoulder 234 can be no more than about 4 GPa or can be as low as 1 GPa, and the modulus of the long tapered stem 236 can be no more than about 4 GPa or can be as low as 0.3 GPa. The modulus of the long tapered stem 236 would ideally transition in a uniform manner from matching the modulus of the shoulder 234 in the area adjacent to the shoulder 234 to a much lower modulus near the end of the long taper stem 236.

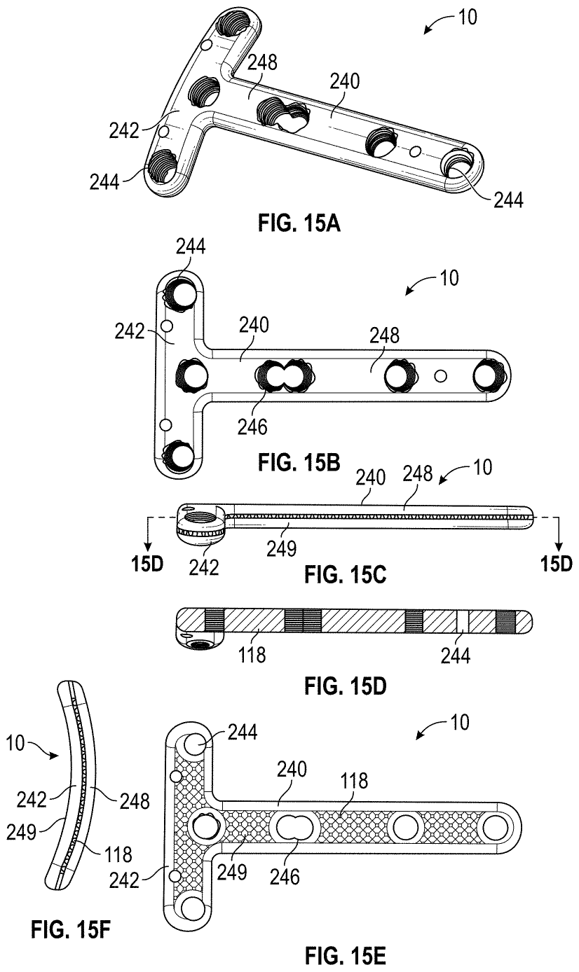

Referring to FIGS. 15A-15F an embodiment of the osteogenesis scaffold 10 with a matrix of arcuate struts 118 is shown. The matrix of arcuate struts 118 is configured in the shape of a fracture repair plate for fixing a fracture bone. It is comprised of a body portion 240, a head portion 242, multiple threaded holes 242, and a double threaded hole 246. The top surface 248 of the body portion 240 and head portion 242 are solid and smooth. The bottom surface 249 of the body portion 240 and head portion 242 is comprised of exposed matrix of arcuate struts 118.

The top portion 28 is smooth as this portion contacts adjacent soft tissue in the patient. The bottom portion 249 has exposed matrix of arcuate struts 118 as this contacts the fractured bone and provides a surface for bone ingrowth as was described previously herein.

Both the body portion 240 and head portion 242 can contain several holes 244 for screws to pass through and into separate portions of fractured bone. In this manner, the osteogenesis scaffold 10 provides fixation between the various portions of the fractured bone. The holes 244 can be threaded as shown or they can be smooth with the head of the screw providing force against the top surface 248 of the osteogenesis scaffold 10. Double holes 246 can also be provided on the body portion 240 (as shown) or the head portion 242 to provide for screws that interfere or lock against each other.

As is the case in other osteogenesis scaffold 10 described herein, the matrix of arcuate struts 118 can vary in stiffness such that the osteogenesis scaffold 10 is stiffer at the main fracture zone and it is more flexible at the ends of the body portion 240 and head portion 242 that are spaced apart from the fracture zone and attached to more flexible bone.

In this manner an osteogenesis scaffold 10 can be provided that fixates fractured bone for healing but does not lead to secondary fractures due to portions of the osteogenesis scaffold 10 that are stiffer than the adjacent healthy bone as is often the case with existing fracture repair plates. As was previously described herein, the variation in stiffness can be achieved using variations in arcuate strut thickness, height, arc length and/or density. For an osteogenesis scaffold 10 that is used for fracture repair, the modulus variation can take more complex forms such as for a multiple point fracture, where the fracture repair plate is stiff at each fracture location and more flexible as the distance from that fracture location increases.

In general, the implant 10 is formed as a cage having a unitary body, with openings provided through the top and bottom surfaces to form cavities or passageways throughout, wherein openings from the top surface are in communication with openings from the bottom surface and are configured and dimensioned to receive graft material, such as bone particles or chips, demineralized bone matrix (DBM), paste, bone morphogenetic protein (BMP) substrates or any other bond graft expanders, or other substances designed to encourage bone ingrowth into the cavities to facilitate the fusion. Additionally the implant 10 may be provided with side openings as shown that are also in communication with the interior cavities.

The implant 10 may be made from any of a variety of materials well known in the orthopedic implant arts. For example, implants may be made from PEEK (polyetheretherketone) such as by being machined therefrom, but alternatively, may be manufactured by injection molding or three-dimensional lithographic printing, for example. When manufactured by three-dimensional lithographic printing, implant 10 may be made of polymers, such as PEEK or other polymer and/or absorbable materials such as tri-calcium phosphate (TCP), hydroxyapatite (HA) or the like. When made of metal, implant 10 may be machined or made by metal powder deposition, for example. Alternatively, implant 10 may be made of PEKK (poly(oxy-p-phenyleneisophthaloyl-phenylene/oxy-p-phenylenetere-phthaloyl—p-phenylene) or carbon-filled PEEK. Manufacturing the implant from any of these materials make it radiolucent, so that radiographic visualization can be used to view through the implant 10 to track the post-procedural results and progress of the fusion over time. Alternatively, implant 10 could be made of titanium or other biocompatible, radiopaque metal. However, this is less preferred as this type of implant would obscure post-procedural radiographic monitoring.

Preferably, the implant comprises a metal such as titanium or a titanium alloy, manufactured using a 3D printing technology. Such technologies are known in several variations, sometimes referred to as Additive manufacturing, rapid prototyping, solid free form technology, powder bed fusion, in which a bed of powdered metal is selectively fused (through sintering or melting) by a laser or electric arc. Also, electron beam melting of metal powder (EBM) may be used.

The embodiments described in the preceding disclosure are generally comprised of a matrix of arcuate struts 118 that have strut cross-sectional dimensions (e.g., diameters) in the range of 0.25 mm to 3 mm. In addition the arcs can range from heights of 1 mm to 6 mm and with arc diameters of 1 mm to 4 mm. Other configurations are possible including arc diameters of 1 mm to 20 mm and arc heights of 1 mm to 25 mm. Furthermore micro arc structures can also be used to create osteogenesis scaffold 10 that has a modulus that matches the adjacent bone modulus and that has a modulus that varies throughout the osteogenesis scaffold 10 to match the variation in modulus of the adjacent bone. These micro structures may be comprised of a matrix of arcuate struts 118 that have strut cross-sectional diameters in the range of 75 to 300 microns and arc heights of 200 to 1,000 microns and arc diameters of 150 to 800 microns.

The three-dimensional lattice configuration of the present invention, including configurations constructed from a plurality of arcuate struts may be adapted for use in a variety of orthopedic applications outside of the spine. For example, intramedullary nails for use in long bones such as the femur, tibia, fibula, radius or ulna may be constructed using the arcuate struts of the present invention, to provide an anisotropic characteristic such as modulus, to match that of the native surrounding environment. Extra medullary implants, such as plates, screws, spacers, rods, sacroiliac joint fusion implants or others may also be constructed utilizing the 3D printed arcuate strut or lattice configurations disclosed here in.

The implants disclosed herein may be provided with a porous or textured surface, such as to facilitate osteogenesis or in the case of porous surfaces, to elute drugs such as antibiotics, anticoagulants, bone growth factors or others known in the art.

Implants produce in accordance with the present invention may alternatively comprise hybrid constructs, with a first component made from 3-D printed lattice and a second component molded, machined or otherwise formed from a conventional implant material such as titanium, various metal alloys, PEEK, PEBAX or others well known in the art.

What is claimed is:

1. An osteogenesis scaffold configured for sacroiliac fusion, comprising:
an elongate body having a proximal end, a distal end and a triangular transverse cross section;
a matrix of arcuate struts on the body providing the body with at least one functional modulus;
wherein the struts are configured to give the body a first modulus at a midpoint between the proximal end and the distal end, that approximates the modulus of cortical bone, and a second, lower modulus at the proximal end and distal end, that approximates the modulus of cancellous bone.

2. An osteogenesis scaffold as in claim 1, wherein a transverse cross sectional area through the body decreases as the cross section approaches at least one of the proximal and distal ends.

3. An osteogenesis scaffold as in claim 2, wherein the first modulus is about 14 to about 16 GPa.

4. An osteogenesis scaffold as in claim 2, wherein the second modulus is about 2 to about 3 GPa.

5. An osteogenesis scaffold configured for spinal fusion, comprising:
a superior support surface;
an inferior support surface;
a plurality of arcuate struts separating the superior and inferior support surfaces, the struts comprising a material having a strut modulus;
the scaffold having a functional modulus which is different than the strut modulus and is the result of the strut modulus and the architecture of the implant.

6. An osteogenesis scaffold as in claim 5, wherein the functional modulus is within the range of from about 5 GPa to about 75 GPa.

7. An osteogenesis scaffold as in claim 5, wherein the functional modulus is within the range of from about 10 GPa to about 30 GPa.

8. An osteogenesis scaffold as in claim 5, wherein the functional modulus in a first portion of the implant is different than the functional modulus in a second portion of the implant.

9. An osteogenesis scaffold as in claim 5, comprising a lumbar cage, having a peripheral modulus within the range of from about 7 GPa to about 16 GPa.

10. An osteogenesis scaffold as in claim 9, comprising a lumbar cage configured for use with normal bone, having a peripheral modulus within the range of from about 14 GPa to about 16 GPa.

11. An osteogenesis scaffold as in claim 8, wherein the functional modulus in a periphery of the superior support surface is greater than the functional modulus in the center of the superior support surface.

12. An osteogenesis scaffold as in claim 11, wherein the functional modulus in a periphery of the superior support surface is at least about 300% greater than the functional modulus in the center of the superior support surface.

13. An osteogenesis scaffold as in claim 12, wherein the functional modulus in a periphery of the superior support surface is at least about 500% greater than the functional modulus in the center of the superior support surface.

14. A proximal femoral osteogenesis scaffold, comprising:
a head post for receiving a femoral head;
a shoulder;
a femoral stem connected to the shoulder;
wherein an exterior surface of the shoulder comprises a smooth surface and the surface of the femoral stem comprises a matrix of struts.

15. A proximal femoral osteogenesis scaffold as in claim 14, wherein the femoral stem has a proximal end in the direction of the shoulder and a distal end, and an increased flexibility in the distal direction.
